(19) United States
(12) Patent Application Publication
(10) Pub. No.: US 2005/0245742 A1
(43) Pub. Date: Nov. 3, 2005

(54) Process for the preparation of zaleplon

(75) Inventor: Stanislav Radl, Praha (CZ)

Correspondence Address: OBLON, SPIVAK, MCCLELLAND, MAIER & NEUSTADT, P.C., 1940 DUKE STREET, ALEXANDRIA, VA, 22314, US

(73) Assignee: ZENTIVA, A.S., Praha (CZ)

(21) Appl. No.: 11/111,818

(22) Filed: Apr. 22, 2005

Related U.S. Application Data
(63) Continuation of application No. PCT/CZ03/00057, filed on Oct. 21, 2003.

(30) Foreign Application Priority Data
Oct. 25, 2002 (CZ) PV 2002-3575

(51) Int. Cl. C07D 487/04

(52) U.S. Cl. 544/281

(57) ABSTRACT

N-[3-(3-Dimethylamino-acryloyl)-phenyl]-N-ethyl-acetamide is reacted with 5-amino-1H-pyrazol-4-carbonitrile in a medium comprising a solution of hydrochloric or hydrobromic acid in a C₁-C₅ alcohol, or in a C₄-C₆ aliphatic or cyclic ether, or in a C₂-C₅ alkoxyalcohol, or in a 5- to 6-membered aromatic heterocyclic solvent, containing 1-2 oxygen atoms, or in mixtures thereof with water, thus forming N-(3-(3-cyanopyrazolo[1,5-α]pyrimidin-7-yl)phenyl)-N-ethylacetamide (zaleplon) of formula I.

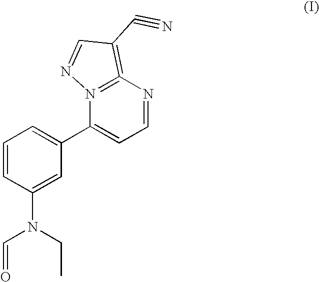

### BACKGROUND OF THE INVENTION

1. Field of the Invention

The present invention relates to a method of producing N-(3-(3-cyanopyrazolo[1,5-α]pyrimidin-7-yl)phenyl)-N-ethylacetamide, known under the International Non-Proprietary Name (INN) of zaleplon, of formula I

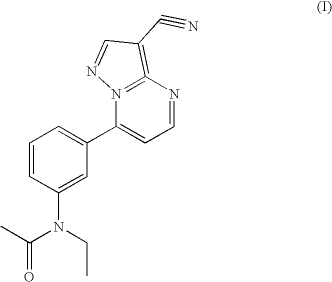

The drug is an important representative of CNS chemotherapeutics, and it is used mainly as an anxiolytic, antiepileptic, or sedative/hypnotic drug.

2. Description of the Background

Zaleplon can be produced by methods such as described in U.S. Pat. Nos. 4,626,538 and 5,714,607 via reaction of N-[3-(3-dimethylamino-acryloyl)-phenyl]-N-ethyl-acetamide of formula II 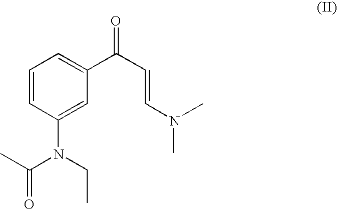
with 5-amino-1H-pyrazol-4-carbonitrile of formula III

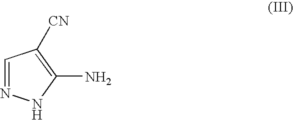

U.S. Pat. No. 4,626,538 describes the original reaction which was conducted in anhydrous acetic acid. Later, it was discovered that the reaction is faster and the resulting product is purer if the reaction is carried out in aqueous acetic acid. This procedure is described in U.S. Pat. No. 5,714,607.

Another process of preparation of zaleplon (I) is disclosed in U.S. Patent Publication 2002/072605, in which compound (II) is formed from the corresponding acetanilide using sodium hydride and ethyliodide in DMF and the reaction mixture is worked-up by dilution with water. The mixture that is formed, without isolation of (II), is treated with a solution of (III) in aqueous DMF in the presence of hydrochloric acid to form (I).

The reaction that is described in patent application WO 02/100828 is one in which the reaction of compound (II) with (I) is conducted in water and a water miscible solvent free of carboxylic groups under acidic conditions.

### SUMMARY OF THE INVENTION

An object of the present invention is to provide and improved method of producing N-(3-(3-cyanopyrazolo[1,5-α]pyrimidin-7-yl)phenyl)-N-ethylacetamide (zaleplon).

Briefly, this object and other objects of the present invention as hereinafter will become more readily apparent can be attained in a method of producing N-(3-(3-cyanopyrazolo[1,5-a]pyrimidin-7-yl)phenyl)-N-ethylacetamide (zaleplon) by reacting N-[3-(3-dimethylamino-acryloyl)-phenyl]-N-ethyl-acetamide of formula II 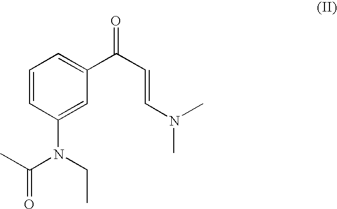
with 5-amino-1H-pyrazol-4-carbonitrile of formula III 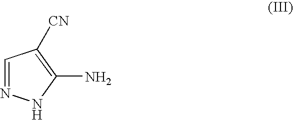
in a medium comprising a solution of hydrochloric or hydrobromic acid in a C₁-C₅ alcohol, or in a C₄-C₆ aliphatic or cyclic ether, or in a C₂-C₅-alkoxyalcohol, or in a 5- to 6-membered aromatic heterocyclic solvent, containing 1-2 oxygen atoms.

### DETAILED DESCRIPTION OF THE PREFERRED EMBODIMENTS

The discovery of the invention is the surprising finding that it is advantageous to perform the reaction of N-[3-(3-dimethylamino-acryloyl)-phenyl]-N-ethyl-acetamide (II) with 5-amino-1H-pyrazol-4-carbonitrile (III) in numerous organic solvents or alternatively in their mixtures with water with a number of organic and inorganic acids being used as catalysts. It has been shown that likewise when aqueous acetic acid or aqueous formic acid is used as catalsyt, the reaction time gets significantly shorter when the above solvents and the above acid catalysts are used. Moreover, in some cases, better yields and/or higher purity of the crude product were achieved.

In a usual embodiment, the two starting substances are mixed with a suitable solvent at room temperature. The quantity of solvent employed can be selected over a wide range so that after the reaction is complete and cooled down, crystals of the product precipitate in high yields. The reaction mixture is either a homogenous solution or a suspension. A suitable acidic catalyst is added to the mixture created in this way and the mixture is stirred at a temperature ranging from 25° C. to the boiling point of the solvent employed. After the reaction was completed, the mixture is cooled down. After crystals precipitate, the mixture is left to sit for several hours at 5 up to 10° C. Subsequently, the crude product is isolated. In some cases, the reaction mixture is mixed with a suitable co-solvent after it is cooled down and the product is filtered after cooling.

A number of solvents are suitable for the reaction. Suitable solvents especially include methanol, ethanol, propanol and 2-propanol, because high yields of highly pure crude product are obtained. The crude product that is obtained is a product of high HPLC purity, usually higher than 99.5% (read from areas of individual peaks) after a single crystallization from a suitable solvent. However, the reaction is not limited only to the environment of hydroxylated solvents. The reaction can be performed in a number of other solvents, for example, tetrahydrofuran, dioxane, dimethoxyethane, or the like. These solvents can be used either alone or in admixtures with the above-mentioned hydroxylated solvents, or in mixtures with water. The presence of 20 to 80% of water is preferable.

In order for the reaction to proceed successfully, the presence of acidic catalysts is necessary. Instead of aqueous acids, solutions of acids in suitable solvents can be used, for example, solutions of hydrogen chloride or hydrogen bromide in various solvents, C₁-C₅ alcohols being preferable.

The invention is explained in more detail in the following examples. The examples, which illustrate preferred alternatives of production of zaleplon according to the invention, have a purely illustrative character and do not limit the extent of the invention in any respect.

### EXAMPLE 1

A mixture of compound II (2.6 g, 10 mmol) and nitrile III (1.1 g, 10.2 mmol) was dissolved in ethanol (30 ml) at room temperature and then, a saturated solution of hydrogen chloride in ethanol (1 ml) was added to the mixture. The resulting mixture was boiled for 2 hours using a reflux condenser and, then, cooled to 5 to 10° C. The product precipitated as crystals and were isolated, washed with water and air dried. An amount of 2.6 g of crude product (85%), m.p. 184-188° C. was obtained. After crystallization, 2.4 g (79%) of crystals were obtained having a m.p. 187-188° C.

### EXAMPLE 2

Following the procedure described in Example 1, wherein the reaction mixture was stirred for 2 days at room temperature, a similar amount of a product was obtained that has a m.p. 182-186° C. After crystallization from methanol, 72% of crystals of product were obtained having a m.p. 185-187° C.

### EXAMPLE 3

A mixture of compound II (2.6 g, 10 mmol) and nitrile III (1.1 g, 10.2 mmol) was dissolved in methanol (30 ml) at room temperature and, subsequently, concentrated hydrochloric acid was added (1 ml) to the mixture. The resulting mixture was boiled for 5 hours using a reflux condenser and, then, water was added (50 ml) and the mixture was cooled to 5 to 10° C. Crystals of product precipitated and were isolated, washed with water and air dried. An amount of 2.8 g of crude product was obtained (92%) having a m.p. of 185-188° C.

### EXAMPLE 4

Following the procedure described in Example 1, propanol was used as the solvent instead of ethanol. A yield of 86% of the product was obtained having a m.p. of 185-188° C.

### EXAMPLE 5

Following the procedure described in Example 1, 2-propanol was used as the solvent instead of ethanol. A yield of 91% of the product was obtained having a m.p. of 184-187° C.

### EXAMPLE 6

Following the procedure described in Example 1, 2-methoxyethanol was used as the solvent instead of ethanol. A yield of 76% of the product was obtained having a m.p. of 184-187° C.

### EXAMPLE 7

Following the procedure described in Example 1, 2-ethoxyethanol was used as the solvent instead of ethanol. A yield of 79% of the product was obtained having a m.p. of 184-187° C.

### EXAMPLE 8

Following the procedure described in Example 1, 70% of aqueous ethanol was used as the solvent instead of ethanol. A yield of 87% of the product was obtained having a m.p. of 185-188° C.

### EXAMPLE 9

Following the procedure described in Example 1, 50% of aqueous ethanol was used as the solvent instead of ethanol. A yield of 69% of the product was obtained having a m.p. of 184-187° C.

### EXAMPLE 10

Following the procedure described in Example 1, wherein 30% of aqueous ethanol was used as the solvent instead of ethanol. A yield of 72% of the product was obtained having a m.p. of 184-188° C.

### EXAMPLE 11

A mixture of compound II (0.26 g, 1 mmol) and nitrile III (0.11 g, 1.0 mmol) was dissolved in tetrahydrofuran (3 ml) at room temperature and, subsequently, concentrated hydrochloric acid was added (1 ml) to the mixture. The resulting mixture was boiled for 2 hours using a reflux condenser and, then, water was added (5 ml) and the mixture was cooled to 5 to 10° C. Crystals of product precipitated and were isolated, washed with water and air dried. An amount of 0.25 g of crude product was obtained (82%), m.p. 184-187° C.

### EXAMPLE 12

Following the procedure described in Example 11, dioxane was used as the solvent instead of tetrahydrofuran and hexane was added instead of water. After the reaction was completed. A yield of 86% of the product was obtained having a m.p. 183-187° C.

### EXAMPLE 13

Following the procedure described in Example 1, hydrobromic acid was used as the catalyst instead of hydrochloric acid. A yield of 81% of the product was obtained having a m.p. 184-188° C.

### EXAMPLE 14

Following the procedure described in Example 1, 10% of sulfuric acid was used as the catalyst instead of hydrochloric acid. A yield of 82% of the product was obtained after thorough washing with water and drying. The product had a m.p. of 184-187° C.

### EXAMPLE 15

Following the procedure described in Example 1, 10% of orthophosphoric acid was used as the catalyst instead of hydrochloric acid and the reaction time was increased to 8 hours. A yield of 65% of the product was obtained after thorough washing with water and drying. The product had a m.p. of 182-186° C.

### EXAMPLE 16

Following the procedure described in Example 1, perchloric acid was used as the catalyst instead of hydrochloric acid. A yield of 69% of the product was obtained after thorough washing with water and drying. The product had a m.p. of 184-187° C.

### EXAMPLE 17

A mixture of compound II (0.26 g, 1 mmol) and nitrile III (0.11 g, 1.0 mmol) was dissolved in ethanol (3 ml) at room temperature and, subsequently, acetic acid was added (1 ml). The resulting mixture was boiled for 3 hours using a reflux condenser and, then, the mixture was cooled to 5 to 10° C. Crystals of product precipitated and were isolated, washed with water and air dried. An amount of 0.26 g of crude product was obtained (85%), m.p. 184-187° C.

### EXAMPLE 18

A mixture of compound II (0.26 g, 1 mmol) and nitrile III (0.11 g, 1.0 mmol) was dissolved in methanol (3 ml) at room temperature and, subsequently, trifluoroacetic acid was added (0.1 ml) to the mixture. The resulting mixture was boiled for 1 hours using a reflux condenser and, then, the mixture was cooled to 5 to 10° C. Crystals of product precipitated and were isolated, washed with water and air dried. An amount of 0.24 g of crude product was obtained (79%), m.p. 184-187° C.

### EXAMPLE 19

Following the procedure described in Example 1, methanesulfonic acid was used as the catalyst instead of hydrochloric acid. A yield of 83% of the product was obtained after thorough washing with water and drying. The product had a m.p. 183-186° C.

### EXAMPLE 20

Following the procedure described in Example 1, benzenesulfonic acid was used as the catalyst instead of hydrochloric acid. A yield of 66% of the product was obtained after thorough washing with water and drying. The product had a m.p. 183-186° C.

### EXAMPLE 21

Following the procedure described in Example 1, 4-toluenesulfonic acid was used as the catalyst instead of hydrochloric acid. A yield of 59% of the product was obtained after thorough washing with water and drying. The product has a m.p. 184-187° C.

The disclosure of Czech patent application Serial No. PV 2002-3575 filed on Oct. 25, 2002 is hereby incorporated by reference into the present application.

Obviously, numerous modifications and variations of the present invention are possible in light of the above teachings. It is therefore to be understood that within the scope of the appended claims, the invention may be practiced otherwise than as specifically described herein.

1. A method of producing N-(3-(3-cyanopyrazolo[1,5-α]pyrimidin-7-yl)phenyl)-N-ethylacetamide, having formula I, comprising: 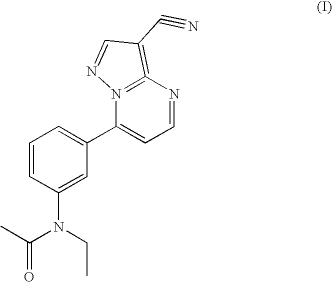
reacting N-[3-(3-dimethylamino-acryloyl)-phenyl]-N-ethyl-acetamide of formula II 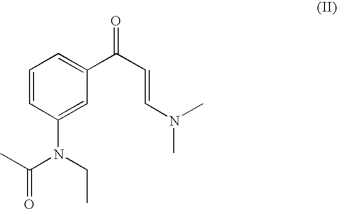
with 5-amino-1H-pyrazol-4-carbonitrile of formula III CN 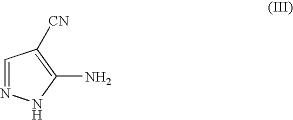
in a medium comprising a solution of hydrochloric or hydrobromic acid in a solvent of a C₁-C₅ alcohol, or in a C₄-C₆ aliphatic or cyclic ether, or in a C₂-C₅ alkoxyalcohol, or in a 5- to 6-membered aromatic heterocyclic compound that contains 1-2 oxygen atoms.

2. The method according to claim 1, wherein ethanol is the solvent.

3. The method according to claim 1, wherein methanol is the solvent.

4. The method according to claim 1, wherein propanol is the solvent.

5. The method according to claim 1, wherein 2-propanol is the solvent.

6. The method according to claim 1, wherein 2-methoxyethanol is the solvent.

7. The method according to claim 1, wherein 2-ethoxyethanol is the solvent.

8. The method according to claim 1, wherein tetrahydrofuran is the solvent.

9. The method according to claim 1, wherein dioxane is the solvent.

10. The method according to claim 1, wherein the solvent for the reaction is a mixture of at least two solvent compounds with water.

11. The method according to claim 1, wherein the solvent for the reaction is a mixture of at least two solvent compounds with 20 to 80% of water.
